(19) United States
(12) Patent Application Publication
(10) Pub. No.: US 2024/0134179 A1
(43) Pub. Date: Apr. 25, 2024

(54) Methods And Systems For High-Resolution And High Signal-To-Noise Ratio Imaging Through Generalized Media

(71) Applicant: The Regents of the University of Colorado, a body corporate, Denver, CO (US)

(72) Inventors: Rafael Piestun, Boulder, CO (US); Sakshi Singh, Boulder, CO (US); Simon Labouesse, Boulder, CO (US)

(21) Appl. No.: 18/546,996

(22) PCT Filed: Feb. 18, 2022
(86) PCT No.: PCT/US2022/017103

§ 371 Date: Aug. 18, 2023

Related U.S. Application Data
(60) Provisional application No. 63/151,052, filed on Feb. 18, 2021.

(51) Int. Cl. G02B 21/16; A61B 5/00; G02B 23/24; G02B 23/26

(52) U.S. Cl. CPC G02B 21/16; A61B 5/0068; A61B 5/0071; G02B 23/2469; G02B 23/26

(57) ABSTRACT

Technology is disclosed herein that enhances imaging, sensing, and property detection of objects. In an implementation, a wave radiation source transmits waves through a complex medium towards an object. The complex medium may be engineered or naturally occurring. Wave modulators modulate the waves transmitted through the complex medium. The wave modulators may comprise spatial or temporal modulators. Secondary waves propagate back though the complex medium in response interaction between the waves and the object. Detectors detect wave properties from the secondary waves. A digital processor reconstructs data based on the secondary wave properties.

### RELATED APPLICATIONS

This application is a national phase of International Application No. PCT/US2022/017103 filed on Feb. 18, 2022, and entitled “METHODS AND SYSTEMS FOR HIGH-RESOLUTION AND HIGH SIGNAL-TO-NOISE RATIO IMAGING THROUGH GENERALIZED MEDIA”; which is related to, and claims the benefit of priority to, U.S. Provisional Patent Application No. 63/151,052 filed on Feb. 18, 2021, and entitled “METHODS AND SYSTEMS FOR HIGH-RESOLUTION AND HIGH SIGNAL-TO-NOISE RATIO IMAGING THROUGH GENERALIZED MEDIA”; the entire content of each of which is incorporated herein by reference.

### STATEMENT REGARDING FEDERALLY FUNDED RESEARCH

This invention was made with government support under grant number 1548925 awarded by the National Science Foundation. The government has certain rights in the invention.

### TECHNICAL FIELD

Aspects of the disclosure are related to the field of confocal microscopy and in particular, to Multiview Scattering Scanning Imaging Confocal (MUSSIC) microscopy to perform high signal-to-noise ratio (SNR) imaging of an object through a multimode fiber.

### BACKGROUND

Multimode fibers make excellent ultra-thin endoscopes that can penetrate deep inside the tissue with minimal damage. Multimode fibers allow for tissue imaging using techniques like confocal microscopy. Confocal microscopy has been used for imaging of the cornea, imaging in body cavities using fiber-optic catheters, and skin cancer detection. Confocal microscopy enables the generation of high-contrast images of 2-D sections within bulk tissue. Confocal microscopy utilizes a scanning focal spot to sample small segments of a target object. Backscattered light is filtered using a small pinhole in the scanning spot's conjugate plane, which blocks the out-of-focus light. The pinhole diameter is chosen to be large enough to achieve a desired tradeoff between optical sectioning and signal integrity. However, scattering limits confocal microscopy to imaging depths only up to around 1 millimeter. Unfortunately, these limitations prevent confocal imaging in the deep tissue regime due to the highly scattering nature of tissue and insufficient signal-to-noise ratio (SNR) levels.

### OVERVIEW

Technology is disclosed herein to image through a general medium with improved resolution and signal to noise ratio (SNR). In an implementation, a system comprises a wave radiation source, a complex medium, wave modulators, detectors, and a digital processor. The wave radiation source transmits waves through the complex medium towards an object. The complex medium may be engineered or naturally occurring. The wave modulators modulate the waves transmitted through the complex medium. The wave modulators may comprise spatial or temporal modulators. Secondary waves propagate back though the complex medium in response interaction between the waves and the object. The detectors detect wave properties from the secondary waves. The digital processor reconstructs data based on the secondary wave properties.

In another implementation, a method to image and sense objects is disclosed. The method comprises delivering a wave through the complex medium from the proximal side of the complex medium towards the distal side of the complex medium. The method continues by receiving secondary waves that are generated from the object that propagate back through the complex medium. The method continues by collecting secondary wave properties on the proximal side of the complex medium at multiple locations. The method continues by implementing a reconstruction algorithm to recover images and/or other properties of the object.

This Overview is provided to introduce a selection of concepts in a simplified form that are further described below in the Technical Disclosure. It may be understood that this Overview is not intended to identify key features or essential features of the claimed subject matter, nor is it intended to be used to limit the scope of the claimed subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the disclosure may be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an exemplary Multiview Scattering Scanning Imaging Confocal (MUSSIC) microscopy system to image an object.

FIG. 2 illustrates an exemplary image reconstruction of an object generated by MUSSIC microscopy.

FIG. 3 illustrates chart that depicts image resolution of MUSSIC microscopy.

FIG. 4 illustrates an exemplary image reconstruction process for MUSSIC microscopy.

FIG. 5 illustrates an exemplary operation of a MUSSIC microscopy system to image an object.

FIG. 6 illustrates an exemplary MUSSIC microscopy system to image an object.

FIG. 7 illustrates an exemplary comparison of confocal and MUSSIC image reconstructions.

FIG. 8 illustrates a chart that depicts normalized cross sections of image reconstructions.

FIG. 9 illustrates a chart that depicts root mean square error for confocal images and MUSSIC images.

FIG. 10 illustrates a chart that depicts the error and correlation of confocal and MUSSIC images as a function of pinhole number.

FIG. 11 illustrates exemplary images obtained by confocal and MUS SIC microscopy.

FIG. 12 illustrates a chart that depicts full width half maxima of Point Spread Functions (PSFs) for confocal and MUSSIC image reconstructions.

FIG. 13 illustrates exemplary MUSSIC images reconstructions for different numbers of pinholes.

FIG. 14 illustrates an exemplary comparison of different image reconstructions.

FIG. 15 illustrates a chart that depicts the average cross sections for different image reconstruction types.

FIG. 16 illustrates an exemplary image comparison between MUSSIC microscopy and Single Pixel Image (SPI).

FIG. 17 illustrates an exemplary computing system to implement an image reconstruction process.

### DETAILED DESCRIPTION

The specification describes a generalized framework that, as opposed to existing techniques such as pixel reassignment confocal imaging, image scanning microscopy, and speckle illumination imaging, enables high-SNR high-resolution imaging with or through a highly complex medium even when there is no shift invariance or memory effects. Hence, the general framework is fundamentally different from prior art because it applies to imaging through media or through systems that are not shift invariant and possibly scattering or multiple scattering in nature. It also applies to images through any medium that can be described by a transmission matrix or a scattering matrix. Such mediums or systems can be either an obstacle to the imaging (located between object and observer) or part of an engineered system in which the generalized medium is created by design. The specification focuses on the implementation of the invention for imaging through MMFs but can be applied to any generalized medium or system as described above. Furthermore, it can be applied to multispectral imaging, fluorescence imaging, and nonlinear imaging of various types (multi-photon, second harmonic, Raman, Coherent Anti-Stokes Raman Scattering, etc.). In one implementation, the system collects images of reflected speckle patterns and back-propagates them digitally to the sample plane with the help of the transmission matrix. The method and systems enable imaging with optical sectioning, high contrast, and high resolution, by switching the bucket detector on the proximal side of the generalized medium with a pixelated camera. The technique also applies to imaging when using separate excitation and detection paths (through different generalized media).

Traditional confocal microscopy is a widely used technique that enables optical sectioning for imaging with high contrast from within scattering tissue volumes. Confocal microscopy employs a scanning focal spot to sequentially sample small segments of the object followed by filtering of the backscattered light using a small pinhole in the scanning spot's conjugate plane, which blocks the out-of-focus light. In practice, the pinhole diameter is chosen to be large enough to achieve a desired tradeoff between optical sectioning and signal integrity. Confocal microscopy has been used for clinical studies for imaging of the cornea, imaging in body cavities using fiber-optic catheters, and skin cancer detection. Unfortunately, confocal imaging in the deep tissue regime remains is difficult or otherwise unfeasible due to the highly scattering nature of tissue and insufficient signal-to-noise ratio (SNR) levels.

Multiphoton microscopy comprises another effective approach to achieve optical sectioning with improved penetration depth. Multiphoton microscopy provides intrinsic optical sectioning without needing to filter the backscattered light through a pinhole due to the two-photon or multi-photon effect. Furthermore, the use of long excitation wavelengths helps achieve penetration depths up to 2 mm using long excitation wavelengths or by employing optical clearing. Unfortunately, multiphoton microscopy often requires expensive pulsed lasers. Furthermore, the long wavelength excitation compromises the lateral resolution, and the penetration depth is limited.

A number of endoscopic systems have been proposed to perform deep tissue imaging like single-mode fibers fiber bundles, GRIN lenses, multicore fibers, and Multimode Fibers (MMF). MMFs provide a minimally invasive and efficient endoscope that can relay a high amount of information for a given cross section. Confocal imaging through MMFs have been made by digitally backpropagating from the detector to the object plane and filtering the signal through a virtual pinhole or by means of optical correlation. Confocal imaging through MMFs showed imaging of 2-D samples with optical sectioning and improved contrast. Unfortunately, confocal imaging through MMFs of thick tissue samples remains impractical due to SNR limitations.

Various solutions are presented herein with respect to the above-mentioned problems in traditional imaging technologies. In some examples, Multiview Scattering Scanning Imaging Confocal (MUSSIC), an acronym used to define the imaging framework of this invention, microscopy is performed through MMFs to overcome the SNR limitations present in confocal microscopy through complex media. MUSSIC microscopy employs multiple coplanar virtual pinholes to collect multiple perspectives of the object. The multiple perspectives may be processed and combined to retrieve a high-SNR confocal image. The MUSSIC microscopy approach should not be confused with the Image Scanning Microscopy (ISM) which is used to boost the SNR in confocal microscopy for a shift invariant system and with neglect of the scattered light. Advantageously and in contrast with ISM, MUSSIC microscopy does not require a direct measurement of the images of the scanning focal spots. Moreover, given the transmission matrix of the system, MUSSIC microscopy can be employed for a more general, shift-variant system such as a complex medium. MUSSIC microscopy enables high Signal-to-Noise Ratio (SNR) imaging through a multimode fiber, hence combining the optical sectioning and resolution gain of confocal microscopy with the minimally invasive penetration capability of multimode fibers. The key advance presented here is the high SNR image reconstruction enabled by employing multiple coplanar virtual pinholes to capture multiple perspectives of the object, re-shifting them appropriately and combining them to obtain a high-contrast and high-resolution confocal image. In some examples, the gain in contrast and resolution in MUSSIC microscopy is compared with other imaging methods like traditional confocal microscopy to verify the concept and demonstrate its advantages.

The MUSSIC microscopy method presented herein may also be used to improve confocal-like imaging to achieve super-resolution. Confocal microscopy has the capability to gain a factor of two in the lateral resolution with respect to the diffraction limited resolution based on the Rayleigh criteria. However, achieving this gain in resolution is impractical as it requires using a detection pinhole smaller than the size of the scanning focal spot, which brings down the signal strength below acceptable levels. MUSSIC microscopy may achieve improved resolution by employing multiple small pinholes whose respective signals can be combined to obtain a reconstruction with a higher SNR.

Improvements to imaging resolution through MMFs may be achieved using two photon imaging, saturated excitation, and by employing a multiple scatterer before the fiber. These approaches however come at the cost of expensive short pulse excitation sources, infeasibly high peak power, and/or loss in transmitted light. A parabolic tip design may be used to increase the effective NA however the design reduces the field of view and requires a non-zero working distance. The non-zero working distance results in the endoscope being susceptible to tissue induced light distortions due to index mismatch. MMFs may improve resolution beyond the diffraction limit by assuming sparsity in samples, however it requires SNR levels of the sample higher than those feasible with bio-compatible markers.

The various embodiments presented herein comprise a generalized framework to demonstrate the principle of MUSSIC microscopy through complex media and the theory for SNR and resolution gain. In some examples, MUSSIC microscopy may be performed through an MMF by measuring its Transmission Matrix (TM). The TM may be used to generate focal spots on the far (distal) end of the MMF. As the focal spots scan the object, reflected speckle patterns on the MMF's near (proximal) end are collected. Using the MMF's TM, the speckle patterns may be back propagated to the object plane to virtually access the scanning focal spot fields and implement MUSSIC microscopy using the weighted pixel reassignment. The approach is quite general and is also applicable to endoscopic imaging systems with separate excitation and detection paths. In some examples the SNR, optical sectioning, and resolution of the reconstructed images of MUSSIC microscopy are compared with conventional confocal and single pixel imaging approaches to illustrate differences MUSSIC microscopy and traditional methods.

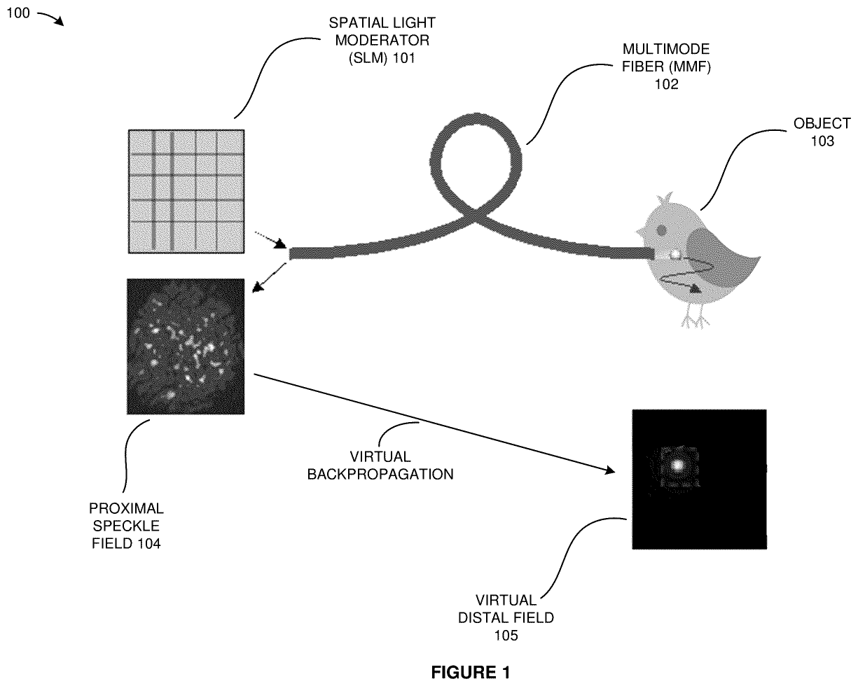

Now referring to the Figures. FIG. 1 comprises Multiview Scattering Scanning Imaging Confocal (MUSSIC) microscopy system 100 to image and/or scan an object of interest. MUSSIC microscopy system 100 is configured to perform tissue imaging, deep tissue imaging, and the like. MUSSIC microscopy system 100 comprises Spatial Light Moderator (SLM) 101, Multimode Fiber (MMF) 102, object 103, proximal speckle field 104, and virtual distal field 105. SLM 101 is positioned at the proximal end of MMF 102 and object 103 is positioned at the distal end of MMF 102. In this example, object 103 comprises a bird however the type of object imaged by MUS SIC microscopy system 100 is not limited. For example, object 103 may comprise an adult human, an infant human, a tissue sample, and/or some other type of suitable object.

In some examples, SLM 101 projects phase patterns to generate scanning focal spots on the distal end of MMF 102 where object 103 is located. For example, SLM 101 may spatially and/or temporally modulate the waves to produce focus spots and/or other patterns on object 103. Object 103 interacts with the phase patterns and reflects light back through MMF 102. The light reflected from object 103 couples back into MMF 102 and reaches the proximal end as speckle field 104. Proximal speckle field 104 is recorded and virtually backpropagated to the distal end using a backward TM. Virtual distal field matrix 105 comprises the MUSSIC raw data.

Imaging through MMF 102 is performed by calibrating the relationship between the input and output fields through the system, described by its Transmission Matrix (TM). The TM of MMF 102 may be measured empirically with both phase and amplitude information by sending an orthogonal set of input fields through MMF 102 accompanied with a phase-stepping reference field. SLM 101 is employed to generate different input fields. The different fields propagating through MMF 102 may be denoted by the letter E followed by different superscripts. MMF 102 has a forward TM denoted T and a distal plane-to-proximal camera plane TM denoted T^{b}.

The set of fields projected on SLM 101 may be described mathematically via matrices. The set of fields may be vectorized and stored in the columns of the matrix denoted Eⁱⁿ. The proximal fields reflected back through MMF 102 may be vectorized and stored in the columns of a matrix denoted E^{p}. The 2D object may be vectorized and stored in the main diagonal of a reflection matrix denoted O. The relationship between the SLM 101, MMF 102, and object 103 may be described using equation 1.

E^{p}=T^{b}OTEⁱⁿ (1)

The subscripts denote the row and column indices of the matrices respectively. If the field illuminating the object is denoted as E^{il}=TEⁱⁿ, then for the k^{th} incident field denoted, E_{*k}ⁱⁿ, where the asterisk indicates the full set of indices along the particular dimension, the l^{th} pixel of the proximal field denoted Eₗₖ^{p} is calculated using equation 2.

Eₗₖ^{p}=Σᵢ₌₁^{N}^{ₒᵤₜ}Tₗᵢ^{b}OᵢᵢEᵢₖ^{il} (2)

Equation 2 shows an overlap function between the excitation and detection Point Spread Functions (PSFs), T_{l*}^{b} and E_{*k}^{il} weighted by the object reflection function O, analogous to the overlap integral used to calculate the resultant field at a confocal pinhole in a conventional confocal microscopy system.

Unlike conventional confocal imaging systems which are shift invariant, the excitation and detection PSFs for MMF 102 follow a complex random distribution. To utilize the raster scan approach for imaging through MMF 102, an input field, E_{*k}ⁱⁿ=T_{*k}^{†} must be projected on SLM 101 to create a diffraction limited focal spot on the k^{th} pixel on the distal end of MMF 102. The dagger denotes the conjugate transpose operation above. Since the detection path is also through MMF 102, the focal spot scanning the object transforms to proximal speckle pattern 104 after reflecting back to the proximal end of MMF 102. The detection path of the reflection destroys some or all spatial information with respect to object 103.

To reverse the effect of the detection path, proximal speckle field 104 is digitally backpropagated to the distal plane using backward TM of MFF 102 to form virtual distal field 105. Virtual distal field 105 may be denoted as E^{d} and is calculated by taking the product of the proximal fields with the inverse of the backward TM. The backward TM typically comprises a poorly conditioned matrix and its inverse may not exist. In such cases where the inverse does not exist, the inverse of the backward TM may be approximated as its conjugate transpose, in a manner similar to the phase conjugated focal spots on the distal end. The backpropagated fields denoted E^{d} can be calculated using equation 3.

E^{d}=(T^{b})^{†}E^{p}=(T^{b})^{†}T^{b}OE^{il} (3)

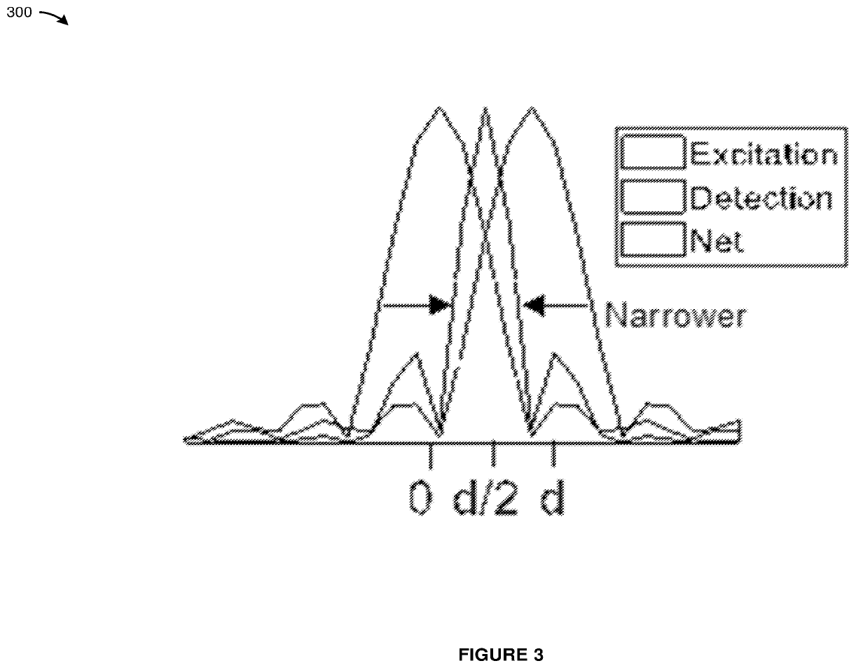

The virtual detection PSF of the system may be defined as D_{T}_{^{b}}=(T^{b})^{†}T^{b}. Similarly, the pre-SLM to distal plane excitation PSF may be defined as, D_{T}=TT^{†}, and includes the wavefront projected on the SLM, T^{†}, for scanning focal spots. Assuming that a plane wave is incident on the SLM, D_{T} is also the illumination field matrix, E^{il}. The matrices D_{T} and D_{T}_{^{b}} have a structure similar to a convolution matrix used to represent the TMs of shift invariant systems. Their Hadamard product yields the net PSF of the system which is narrower than the individual PSFs as depicted in FIG. 3. This narrower net PSF is the source of resolution gain in MUSSIC microscopy system 101. The resolution enhancement depends in part on the size of each virtual pinhole relative to the size of the virtual detection Airy disk.

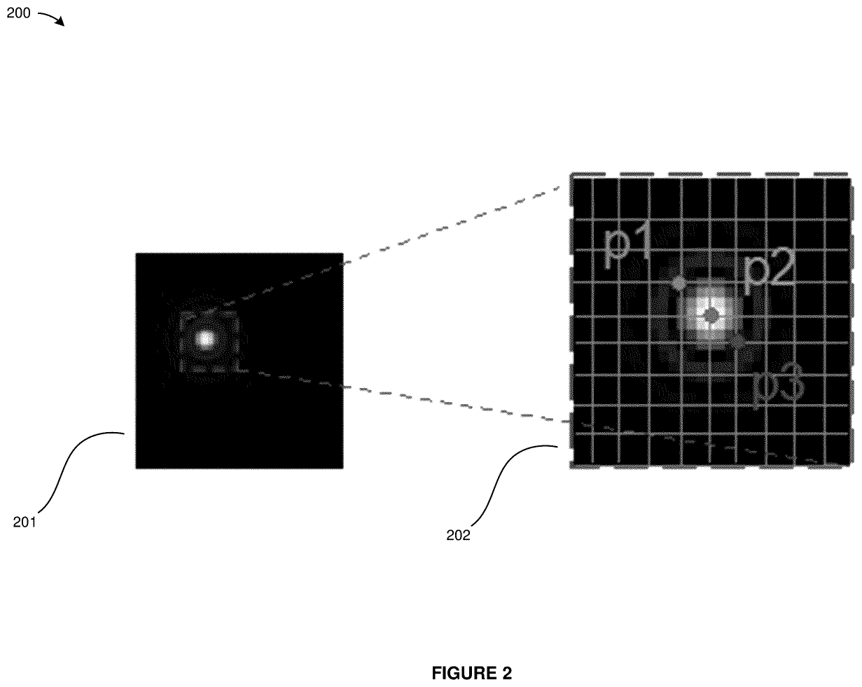

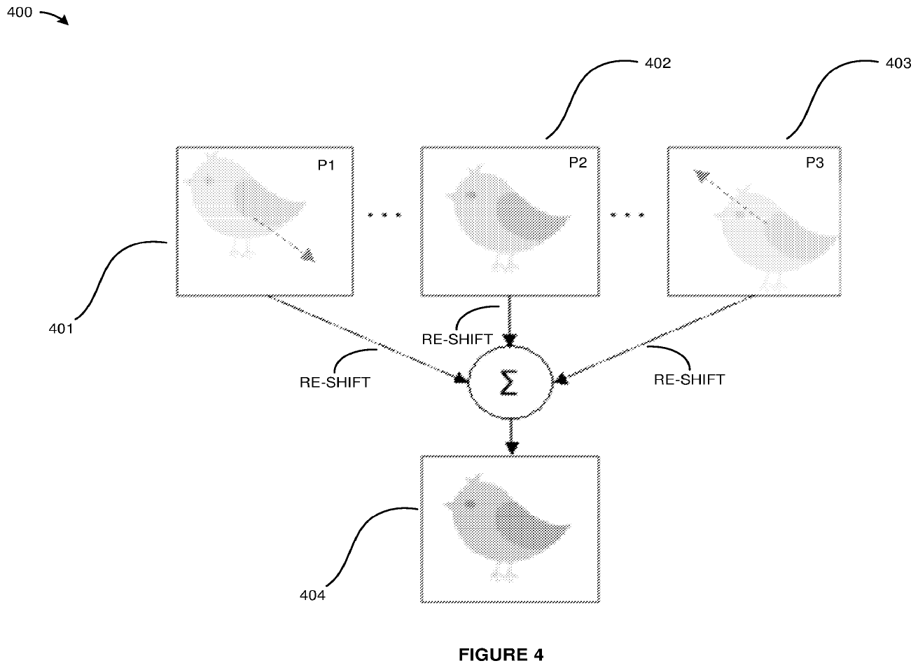

Once the full virtual distal field matrix denoted E^{d} is obtained, the on-axis confocal image is obtained from its main diagonal, Eₖₖ^{d}, where k∈(1, Nᵢₗ) denotes all distal scan positions. This main diagonal comprises the measurements from the central virtual pinhole, p2 indicated in FIG. 2. Similar mutually shifted confocal images are also obtained from the diagonals, Eₗₖ^{d}, corresponding to the neighboring pixels of k (such as p1 and p3 shown in FIG. 1(b)) where l takes N²−1 values other than k in the N×N-pixel neighborhood of each scan position k. All the N² confocal images can then be reshifted to a common axis, weighted in proportion to their signal strength and combined to yield a high-SNR MUSSIC image reconstruction as illustrated in FIG. 4.

Prior to the imaging of object 103, the forward TM of MMF 102 must be calibrated. The forward TM of MMF 103 denoted T may be measured with both phase and amplitude information by sending a complete basis of orthogonal input fields into the fiber accompanied with a phase-stepping reference field. A plane waves basis is chosen that transforms to focal spots in the Fourier plane and may then be coupled into MMF 103. These patterns are constant in amplitude and their phases are modulated using SLM 101. SLM 101's active area is divided into two sections each for the changing grating pattern and a phase-stepping reference frame that surrounds it. The intensity measurements at the fiber output for each projected pattern, as the reference field is phase stepped, allows for the recovery of the output fields. These output fields may then be vectorized and used to build all the rows of the matrix T.

Likewise, the backward TM of MMF 102 must also be calibrated prior to imaging The TM of MMF 102 obeys the reciprocity rule in that T^{b}=T′. Since the TM is recorded between SLM 101 and the distal plane, the above assumption only holds true if the detection plane perfectly matches the SLM plane in scale and orientation. This requirement results in challenging task and may require a sensitive and time-consuming alignment procedure. It also does not account for coupling losses from the sample to the fiber distal end. It is desirable to separate the collection and detection pathways in endoscopes to improve throughput or to gain some feedback from the distal end, in which case T^{b}≠T′. For other modalities like fluorescence imaging, the excitation and detection PSFs are different by default due to difference in the excitation and fluorescence wavelengths. With these considerations, a separate calibration of the matrix T^{b} from the distal plane to the detector plane is needed.

Towards this end, a mirror is placed at the distal end of the fiber and focal spots

are scanned on it. The reflected fields on the proximal end are measured and denoted as E^{p-mirror}. These measurements give result in an estimate of T^{b}, and is denotes as T_{obs}^{b} as described in equation 4.

T_{obs}^{b}=E^{p-mirror}=T^{b}I E^{il} (4)

The matrix I of equation 4 represents the mirror reflection matrix, which is assumed to be an identity matrix. The distal fields are then given by equation 5.

E^{d}=(T_{obs}^{b})^{†}E^{p}=(E^{il})^{†}D_{T}_{^{b}}OE^{il} (5)

The additional rightmost term (E^{il})^{†} on the right-hand side of the above equation occurs because of the double pass approach for calibration of T^{b}. Since a raster scan approach is used, both the E^{il} and (E^{il})^{†} matrices have the structure of a convolution matrix with a diffraction limited Gaussian kernel and E^{d} still gives a measure of the confocal images of the object. The theoretical resolution gain is also preserved as the bandwidth of the terms on the left and right of object 103 denoted, O, in the above equation remain unchanged.

In some examples, the conjugate transpose operator may be used when the inverse of a matrix does not exist. This is useful for generating perfect phase conjugated focal spots, as required when raster scanning on the distal side of the fiber. However, when calculating the backpropagated distal fields, the conjugate transpose is not the best inversion method. The inversion of the backward TM can be optimized using a Tikhonov regularization technique. This technique involves computing the singular value decomposition of the backward TM, T_{obs}^{b}=USV^{†} and finding its inverse using equation (6).

T_{obs}^{b}^{^{RI=VS}}^{RI}U^{†} (6)

S^{RI} comprises the regularized inverse of the diagonal matrix of singular values, S, calculated by replacing the singular values σᵢ in the diagonal of S with

$$\frac{{σ}_{i}}{{σ}_{i}^{2} + {β}^{2}} ,$$

where β is the regularization parameter. By calculating the backpropagated distal fields using Tikhonov regularized inverse of the T_{obs}^{b} instead of (T_{obs}^{b})^{†} in equation 5, image reconstructions with improved SNR and contrast are produced. In this example, a β value equal to 10% of the highest singular value of the backward TM was chosen.

Digital filtering may be performed to bandlimit the acquired data. This eliminates the noise in the high frequencies and ensures that all acquired images have speckles with a minimum grain size limited by diffraction. The frequency cutoff may be found by setting a minimum threshold to the total energy in the frequency space averaged over all acquired images.

The reconstructed confocal and MUSSIC images of object 103 are normalized with respect to their “blank” counterparts. The blank counterparts comprise the confocal and MUSSIC images obtained when a mirror is placed at the distal end in place of object 103. This helps account for the non-uniformity and intensity variations in the focal spots used to scan the object and significantly improves the image quality. The effect is particularly strong since a non-uniform internal reference for phase measurements is employed.

In some examples, object 103 may be imaged without full field backpropagation. In such examples, the calculation of the full matrix E^{d} involves heavy computation, with a complexity O (Nᵢₗ²Nᵢₙ). Access to the full backpropagated distal fields is not necessary to calculate the confocal or MUS SIC images. The only data points required in each distal field are in the neighborhood of the scanning focal spot, for every scan position. This number, which can be defined as Nₚᵢₙₕₒₗₑₛ is chosen to be roughly equal to the number of pixels that sample a focal spot and is lower than the number of illuminations used for imaging. If only the desired diagonals from the matrix E^{d} corresponding to the Nₚᵢₙₕₒₗₑₛ neighboring pixels are computed, the complexity of the calculation drops down to O (NₚᵢₙₕₒₗₑₛNᵢₙNᵢₗ) for the MUSSIC image and O (NᵢₙNᵢₗ) for a single confocal image. When using the conjugate transpose of the backward TM to invert it, the method for obtaining the confocal image is similar to the correlation method.

In some examples, MUS SIC microscopy system 100 may implement a digital optical phase conjugation method to perform virtual backpropagation to generate virtual distal field 105. For example, a processor may receive proximal outputs from MMF 102 and record output patterns of the backpropagating waves with a holographic acquisition.

In some examples, MUSSIC microscopy system 100 may implement an optical correlation process to perform virtual backpropagation to generate virtual distal field 105. For example, a processor may receive proximal outputs from MMF 102 and optically correlate the light signals returning through MMF 102 with an input pattern to generate virtual distal field 105. It should be appreciated that the method used to perform virtual backpropagation is not limited.

Imaging using the correlation method, enables MUSSIC reconstruction of a 20,000-pixel image in 4 minutes on a DELL Desktop computer with a 3.2 GHz Intel Core i5 processor and 64 GB RAM. A comparison of correlation and Tikhonov regularized reconstructions reveals that although the regularization considerably improves the image quality, the faster reconstruction also provides a good estimate of the object.

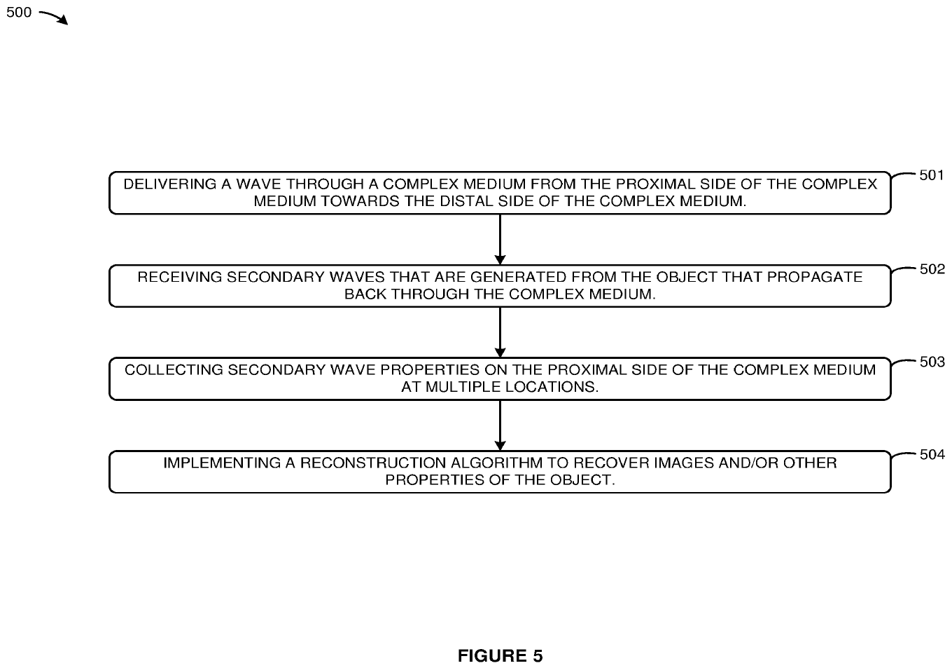

In some examples, MUSSIC microscopy system 100 implements process 500 illustrated in FIG. 5. It should be appreciated that the structure and operation of MUSSIC microscopy system 100 may differ in other examples.

FIG. 2 illustrates image reconstruction 200. Image reconstruction 200 comprises virtual distal field 201 and zoomed in virtual distal field 202. Virtual distal field 201 comprises an image generated by a MUSSIC microscopy system. For example, MUSSIC microscopy system 100 may image object 103 and responsively generate virtual distal field 201. Zoomed in virtual distal field 202 comprises a zoomed in version of virtual distal field 201 and is zoom-in on the focal spot as demarcated by the red dotted line. A discrete grid is overlayed onto zoomed in virtual distal field 202. The intersection points on the grid indicate a data point and can be viewed as a virtual pinhole. Each intersection point of the grid of zoomed in virtual distal image 202 corresponds to a data point or pixel, that acts as a virtual pinhole. While the central pixel, p2 measures the on-axis confocal image, the pinholes p1 and p3 also measure similar confocal images from different perspectives.

FIG. 3 illustrates a chart that depicts resolution improvement resulting from MUSSIC microscopy. The excitation PSF, D_{T}, the virtual detection PSF, D_{T}_{^{b}}, and the net PSF, calculated as the product of the former two are shown respectively for an example confocal image obtained from a virtual pinhole pᵢ at a distance d from the on-axis pinhole. Although the net PSF is shifted from the axis by a distance, d/2, it is narrower than the former two PSFs, hence leading to an improved resolution. The excitation and detection wavelengths are assumed to be identical here.

FIG. 4 illustrates an exemplary image reconstruction process. FIG. 4 comprises environment 400 which comprises confocal images 401-403 and reconstructed image 404. Environment 400 illustrates the process of retrieving a high SNR MUSSIC image from multiple confocal images 401-403 obtained from different virtual pinholes. Confocal images 401-403 respectively correspond to pinholes p1, p2 and p3, as marked FIG. 2. Besides the image obtained from the on-axis pinhole, p2, all confocal images have parallax errors determined by the location of their corresponding virtual pinholes. By applying appropriate shifts and weights to them and combining them together, a single high-contrast MUSSIC image is obtained.

FIG. 5 illustrates exemplary process 500. Process 500 comprises an object imaging process. Process 500 may be implemented in program instructions in the context of any of the software applications, module components, or other such elements of one or more computing devices. For example, a computing device may carry out a virtual backpropagation process with respect to FIG. 1. The program instructions may direct computing device(s) to operate as follows, referred to in the singular for the sake of clarity.

Process 500 beings by delivering a wave through a complex medium from the proximal side of the complex medium towards the distal side of the complex medium (501). Process 500 continues by receiving secondary waves that are generated from the object that propagates back through the complex medium (502). Process 500 continues by collecting secondary wave properties on the proximal side of the complex medium at multiple locations (503). Process 500 continues by implementing a reconstruction algorithm to recover images and/or other properties of the object (504).

Referring back to FIG. 1, MUSSIC microscopy system 100 includes a brief example of process 500 as employed by one or more applications, devices, mechanical, and/or computing systems. In operation, SLM 101 delivers a wave through MMF 102 from the proximal side of MMF 102 where SLM 101 is positioned towards the distal side of MMF 102 where object 103 is positioned (501). For example, SLM 101 SLM 101 may comprise a laser that sends an electromagnetic wave through MMF 102. It should be appreciated that the type of radiation source used to generate the wave is not limited. The wave travels through MMF 102 and interacts with object 103. The interaction between the wave and object 103 generates secondary waves that propagate back through MMF 103 from the distal side where object 103 is positioned towards the proximal side where they are collected (502). For example, the secondary waves may be generated by the reflection of the wave off of object 103. The secondary waves properties are collected on the proximal side of MMF 102 to form proximal speckle field 104 (503). For example, an imaging system may be positioned at the proximal side of MMF 102 to receive the secondary waves propagating back through MMF 102, process the secondary waves to identify the secondary wave properties, and generate proximal speckle field 104 based on the wave properties. A computing device applies a reconstruction algorithm to proximal speckle field 104 to generate virtual distal field 105 via a virtual backpropagation process (504). The virtual backpropagation process recovers images and/or other properties of object 103.

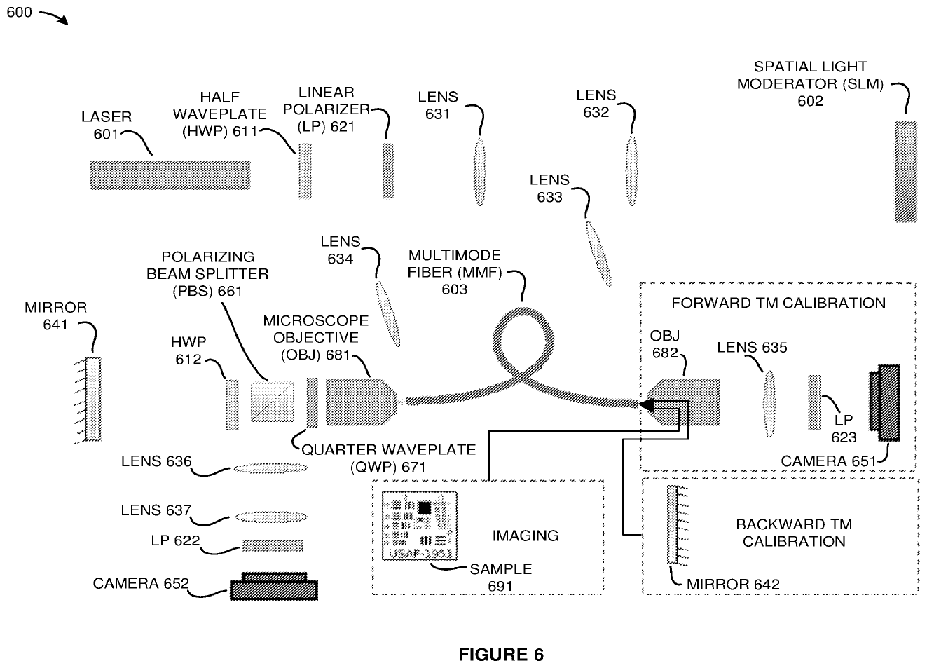

FIG. 6 illustrates MUSSIC microscopy system 600 to perform object imaging. MUSSIC microscopy system 600 comprises an example of MUSSIC microscopy system 100, however system 100 may differ. MUSSIC microscopy system 600 comprises laser 601, Spatial Light Moderator (SLM) 602, Multimode Fiber (MMF) 603, Half Waveplates (HWPs) 611-612, Linear Polarizers (LPs) 621-623, lenses 631-637, mirrors 641-642, cameras 651-652, Polarizing beam Splitter (PBS) 661, Quarter Waveplate (QWP) 671, Microscope Objectives (OBJs) 681-682, and sample 691.

In some examples, laser 601 emits a laser beam to perform forward TM calibration of MMF 603. The laser beam goes through HWP 611 and LP 621. HWP 611 and LP 621 perform polarization control on the laser beam. The beam then travels through a 4-F system comprising lenses 631-632 that narrows the beam diameter to match the active area of SLM 602. Interaction between the beam and SLM 602 forms an SLM plane. The SLM plane of SLM 602 is then imaged onto the back-aperture of OBJ 681 via lenses 633-634 and mirror 641. OBJ 681 couples the couples the light of the SLM plane into MMF 603. PBS 661 is placed between SLM 602 and OBJ 681 to direct the back-reflected light from MMF 603 onto camera 652. HWP 612 is placed between PBS 661 and SLM 602 and allows for the controlling of the polarization axis of the incident beam. QWP 671 is placed between PBS 661 and OBJ 681. QWP 671 along with PBS 661 act as an optical isolator to prevent back-reflections from the proximal facet of the fiber from reaching the camera. The distal facet of MMF 603 is imaged onto camera 651 using lens 635 during the forward TM calibration. LP 623 located between lens 625 and camera 651 allows for the detection of only one polarization component. Camera 651 captures the images of the distal facet of MMF 603. The forward TM matrix is then calibrated based on these measurements.

After the forward TM calibration of MMF 603 is complete, the backward TM calibration of MMF 603 is initiated. Mirror 642 is placed near the fiber distal tip for calibration of the backward TM to reflect emissions from the distal end of MMF 603 back through MMF 603 to calibrate the backward TM. The backward TM of MMF 603 is calibrated using back-reflected fields on the proximal side of MMF 603, while focal spots are projected on the distal side. A phase shifting reference frame is simultaneously projected on SLM 602 along with the phase conjugated patterns for distal raster scan, for measuring both the phase and amplitude of the back-reflected fields. The back-reflected light from mirror 642 couples back into MMF 603 and is detected on the proximal side using camera 652. Camera 652 images the back-aperture of OBJ 681 using another 4-F system comprising lenses 636-637 and is placed in a plane equivalent to the SLM plane of SLM 602. LP 622 is placed before camera 652 and allows for the detection of a single polarization component. The imaging from camera 652 allows for the backward TM calibration of MMF 603.

After both calibrations are complete, mirror 642 is removed and replaced by sample 691. Sample 691 is placed at the distal facet of MMF 603. The back-reflected fields from sample 691 are recorded by camera 652 as sample 391 is raster scanned. In some examples, laser 601 comprises a 785 nm CW Crystal laser. In some examples, SLM 602 comprises a Meadowlark optics liquid crystal SLM (HSPDM 512) for phase modulation. In some examples, MMF 603 comprises a step-index fiber of diameter 50 μm and 0.22 numerical aperture (NA) for all our experiments. Although MMF 603 may comprises a fiber, it should be appreciated the MMF 603 may comprise a variety of engineered or naturally occurring complex mediums. For example, MMF 603 may comprise a metamaterial, a diffractive element, a hologram, a coded aperture, a stratified element, a shift variant system, and the like. Although MUSSIC microscopy system 600 utilizes camera 652, to image sample 691, other types of imaging devices may be used. For example, camera 652 may comprise a photodetector, a photodetector array, a single photon avalanche diode array, a photomultiplier tube, point detectors that are shifted in space, or some other type of suitable imaging device.

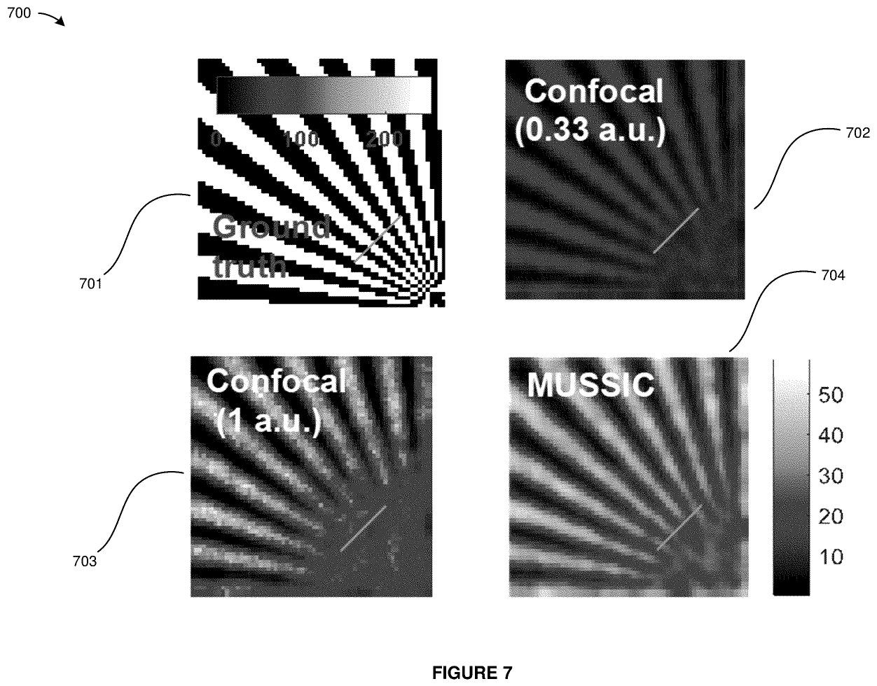

FIG. 7 illustrates environment 700 which comprises an exemplary comparison of confocal and MUSSIC image reconstructions. Environment 700 comprises ground truth image 701, confocal images 702-703, and MUSSIC image 704. Ground truth object 701 comprises an imaged sample in the form of a siemens star. Confocal image 702 comprises a confocal reconstruction of ground truth object 701 using a 3×3 macro-pinhole setup. Confocal image 702 comprises a confocal reconstruction of ground truth object 701 using a 9×9 macro-pinhole setup. MUSSIC image 704 comprises a MUSSIC reconstruction of ground truth object 701 using a of 9×9 pinhole setup, each of radius 0.11 a.u.

In some examples, confocal and MUSSIC microscopy is performed on an object to compare the SNR and resolution of the reconstructed images generated by the different methods. For example, MUSSIC microscopy system 601 may image sample 691 using a MUSSIC method and a confocal method to identify differences in resolution and SNR between the two methods. For the image comparison, the MMF TM is modeled as a complex random matrix and reconstruct the image of a quadrant of a binary Siemens star using simulated proximal speckle fields, following the backpropagation process described in the preceding paragraphs. Gaussian noise with 5% variance is added to the simulated proximal fields before the image reconstruction. Each virtual pinhole in the simulation has a radius of 0.11 Airy unit (a.u.), where we have defined 1 Airy unit as the radius of the Airy disk scanning the object. Hence one Airy disk spans across 9×9 individual pinholes.

The SNR improves significantly between confocal image reconstruction 702 and confocal image reconstruction 703 as the size of the macro-pinhole increases. However, the resolution degrades as the size of the macro-pinhole increases between confocal image reconstruction 702 and confocal image reconstruction 703. MUSSIC image reconstruction 704, which uses the same group of pinholes as confocal image 703 retains high-SNR and also preserves the resolution. Confocal reconstruction 703 with the 1 a.u. pinhole fails to resolve the image features, while MUSSIC reconstruction 704 using the same raw data resolves them just as well as the confocal reconstruction with the 0.33 a.u. pinhole.

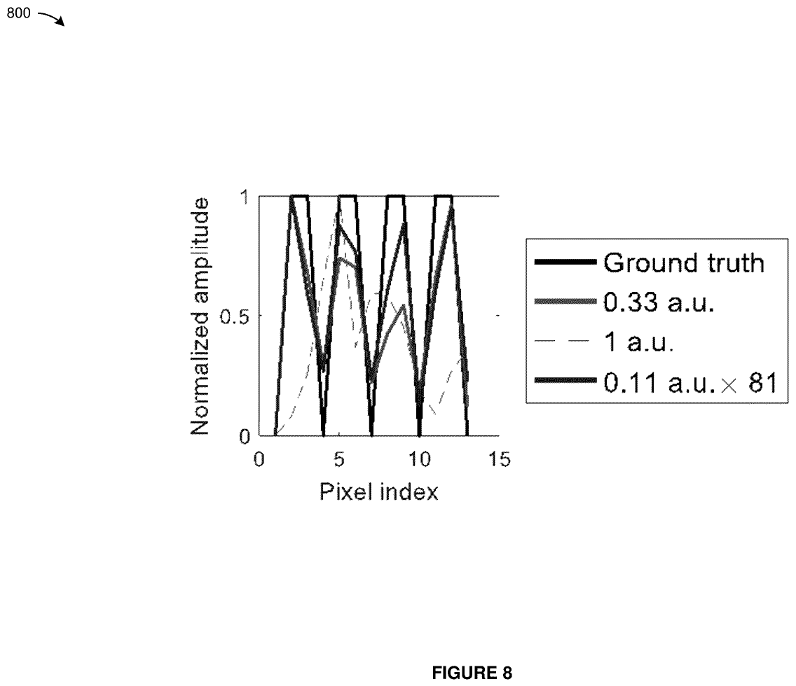

FIG. 8 illustrates chart 800. Chart 800 depicts the relationship between normalized amplitude and pixel index for confocal and MUS SIC image reconstructions. The plot labeled ground truth corresponds to ground truth object 700 illustrated in FIG. 7. The plot labeled 0.33 a.u. corresponds to confocal image reconstruction 702 illustrated in FIG. 7. The plot labeled 1 a.u. corresponds to confocal image reconstruction 703 illustrated in FIG. 7. The plot labeled 0.11 a.u.×81 corresponds to MUSSIC image reconstruction 704 illustrated in FIG. 7. As illustrated in chart 800, the plot the corresponds to the MUS SIC image reconstruction corresponds to the ground truth plot more closely than the confocal reconstruction plots. In some examples, the different plots of chart 800 may correspond to a particular cross section in ground state image 701 and reconstructions 702-704.

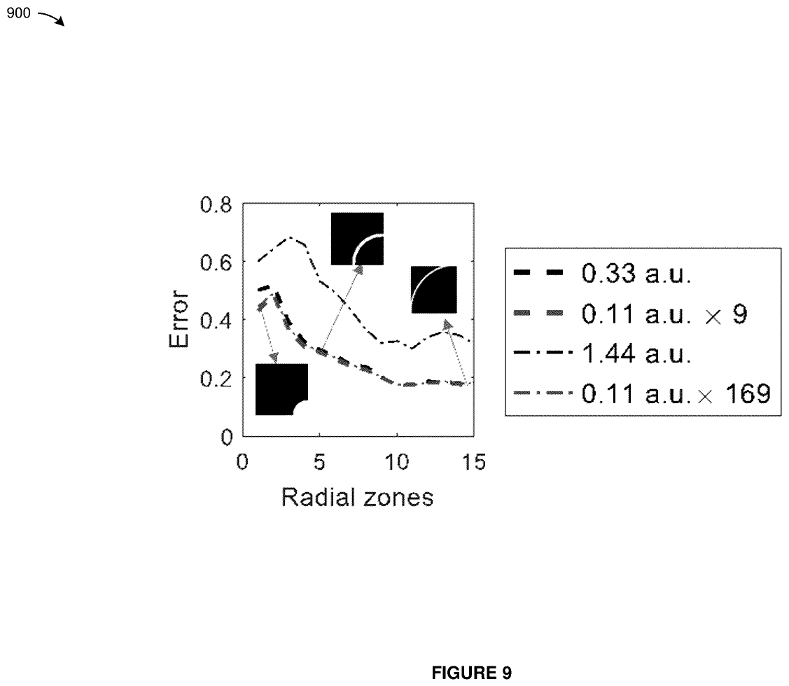

FIG. 9 illustrates chart 900. Chart 900 depicts the relationship between the root mean square error of different normalized reconstructions (i.e., reconstructed images 702-704) as a function of the annular radius measured from the center of the seimens star (i.e., ground truth image 701) or the bottom right corner. For this comparison, no noise was added to the reconstructions to analyze the effect of using increasing number of pinholes on resolution. In this example, the image quadrant is divided into 15 radial zones and the error is plotted with respect to the ground truth image in each zone for the different reconstruction methods. The error for the confocal reconstruction images increases with the radius of the macro-pinhole. The error in the MUS SIC reconstruction remains almost unchanged as the number of used pinholes increases from 3×3 to 13×13. The inset images in the figure show the radial zones 1, 5 and 15 from left to right.

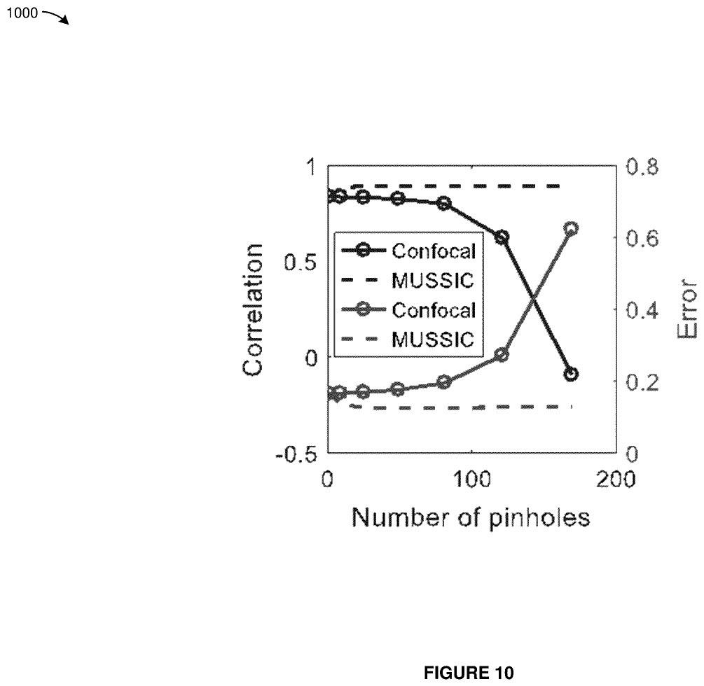

FIG. 10 illustrates chart 1000. Chart 1000 depicts the relationship between the reconstruction error and correlation as a function of the number of pinholes used for a given cross section of images 701-704 in the absence of noise. The error and correlation with respect to the ground truth cross-section increases and decreases respectively as the number of pinholes constituting the macro-pinhole increases for confocal reconstructions (i.e., confocal reconstructions 702-703). On the other hand, both metrics for the MUSSIC reconstructions (i.e., MUSSIC reconstruction 704) remain unaffected, indicating that the image quality is preserved.

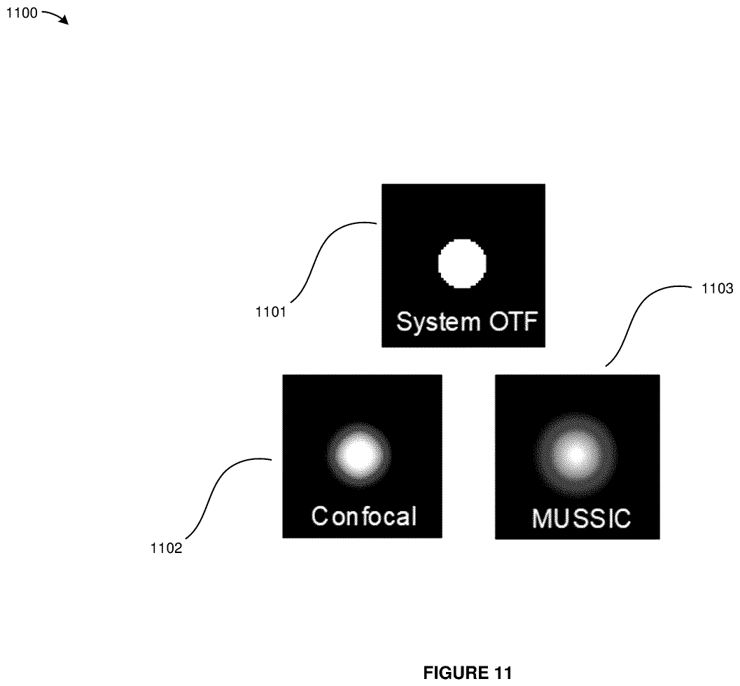

FIG. 11 illustrates environment 1100. Environment 1100 comprises system OTF frequency response 1101, confocal frequency response 1102, and MUSSIC frequency response 1103. The average frequency response for confocal and MUS SIC methods were obtained for a point object using 81 pinholes. These responses were also obtained in the absence of noise. The frequency cutoff of the MUSSIC reconstruction is almost the double that of the Optical Transfer Function (OTF) of the system, which is the theoretically claimed gain in resolution according to Rayleigh's criteria. On the other hand, the confocal reconstruction obtained from the 1 a.u. pinhole has a frequency cutoff 1.4 times higher than that of the system OTF.

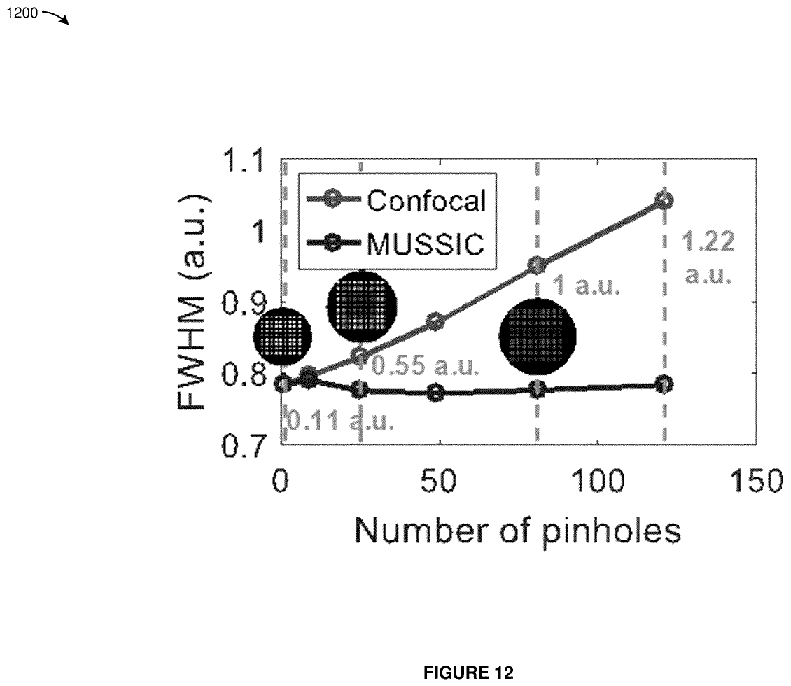

FIG. 12 illustrates chart 1200. Chart 1200 depicts the relationship between the Full Width Half Maxima (FWHM) and the number of pinholes for noiseless Point Spread Functions (PSFs). Noiseless PSFs are computed for the confocal and MUSSIC methods and plotted the FWHM of the PSFs as a function of the number of pinholes used. The FWHM for the confocal reconstruction increases with the number of pinholes constituting a macro-pinhole, while the FWHM for the MUSSIC reconstruction remains unchanged. The net confocal pinhole size in Airy units is indicated at various points. The black circular insets illustrate a 1 a.u. pinhole arrays.

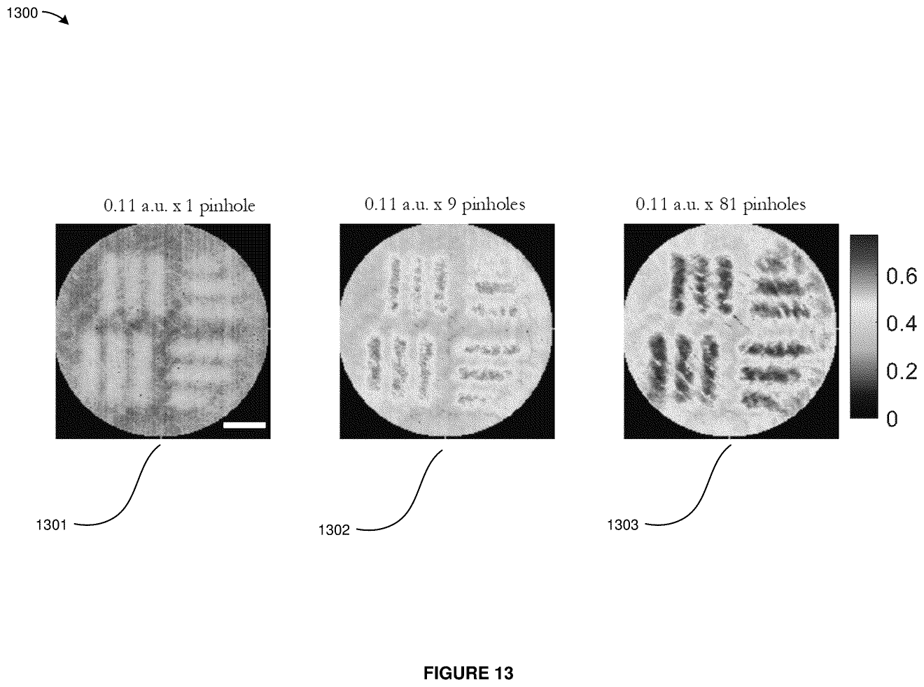

FIG. 13 illustrates environment 1300. Environment 1300 comprises MUSSIC image reconstructions 1301-1303 for various numbers of pinholes. In some examples, the MUSSIC image reconstructions are generated by MUSSIC microscopy through a multimode fiber. The difference in SNR for reconstructions 1301-1303 are shown. In this example, the imaged object comprises the fourth and fifth elements of the 7^{th} group in the USAF 1951 resolution target, which have a resolution of 181- and 203-line pairs/mm respectively. The field of view is a 50 microns wide-160×160-pixel window. MUS SIC reconstructions 1301 was captured using one pinhole, MUSSIC reconstructions 1302 was captured using nine pinholes, and MUSSIC reconstructions 1303 was captured using 81 pinholes. The reconstruction SNR improves with an increase in the number of pinholes.

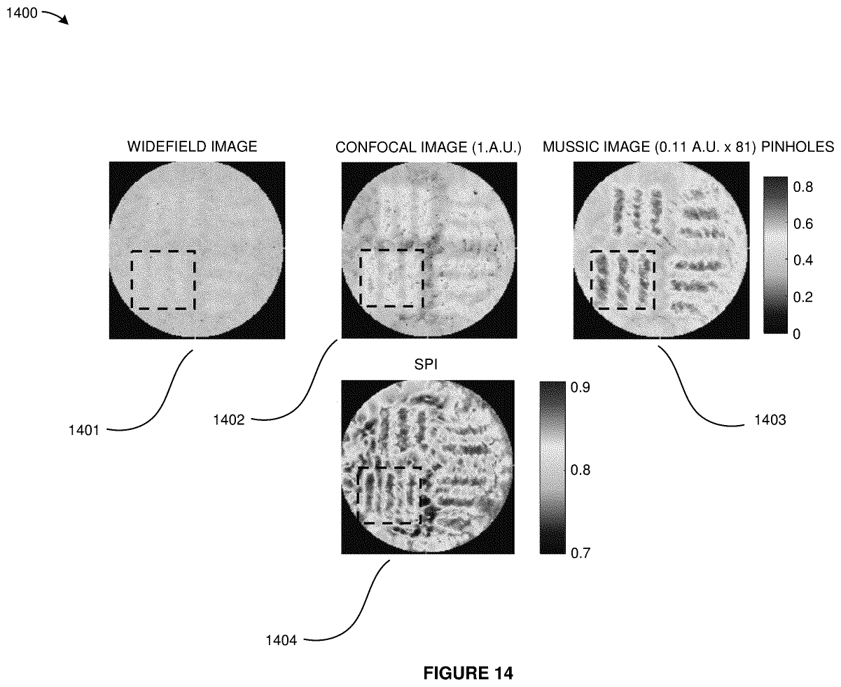

FIG. 14 illustrates environment 1400. Environment 1400 comprises image reconstructions 1401-1404 for various types of reconstruction methods. Reconstruction image 1401 comprises a widefield image obtained by averaging over the computed distal fields. Reconstructed image 1402 comprises a confocal image obtained using 81 pinholes of 1 a.u. radius. Reconstructed image 1403 comprises a MUSSIC image obtained using 81 pinholes of 0.11 a.u. radius. Reconstructed image 1404 comprises an SPI image obtained by integrating the absolute value of the proximal fields. The widefield reconstructed image 1401 is noisy and low in contrast. These problems can be attributed to noise in the computation of the virtual distal fields and the limited enhancement of the scanning focal spots. By integrating only 81 pinholes lying within 1 a.u. of the scanning focal spot, a confocal image reconstruction 1402 with improved contrast is obtained. MUSSIC image reconstruction 1403 using 81 pinholes shows an improved contrast with respect to confocal reconstruction 1402.

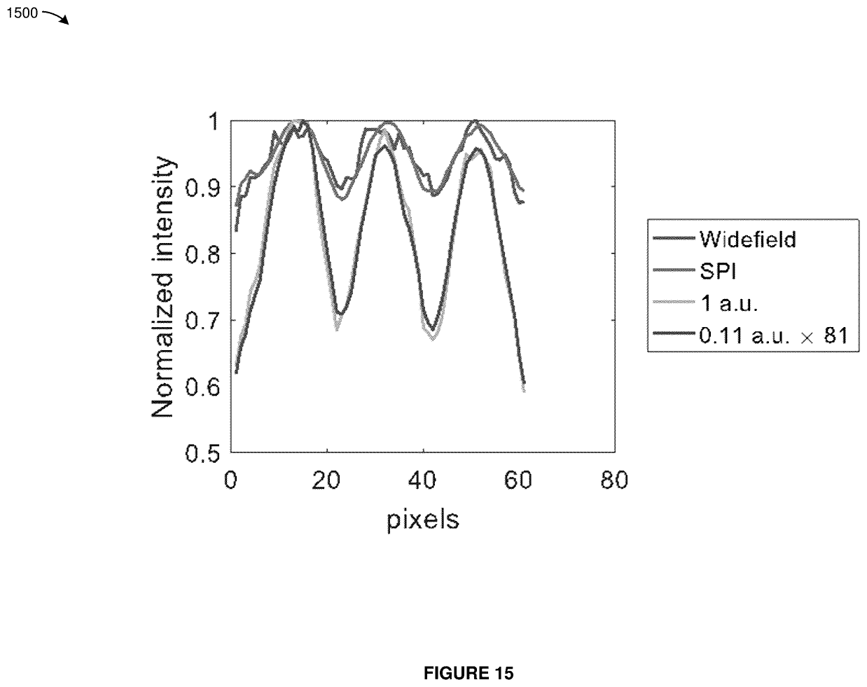

FIG. 15 illustrates chart 1500. Chart 1500 depicts the relationship between of normalized intensity at pixel locations for image reconstructions 1401-1404. Chart 1500 plots the normalized average cross section along the horizontal direction for a cropped window within the FOV indicated using black-dashed lines. Chart 1500 comprises a plot for widefield image reconstruction 1401, confocal reconstruction 1402, MUSSIC reconstruction 1403, and SPI reconstruction 1404. Chart 1500 indicates that confocal reconstruction 1402 and MUSSIC reconstruction 1403 have a much better contrast than widefield reconstruction 1401. Chart 1500 further indicates that SPI reconstruction 1404 which was obtained without virtual backpropagation and using the signal from all the distal pinholes, has a higher SNR than other reconstructions 1401-1403 but lower contrast similar to the widefield image. The cross-sections of the widefield and MUSSIC images have a peak-to-valley modulation depths of 0.12 and 0.25 (normalized intensity units) respectively, showing a significant resolution improvement.

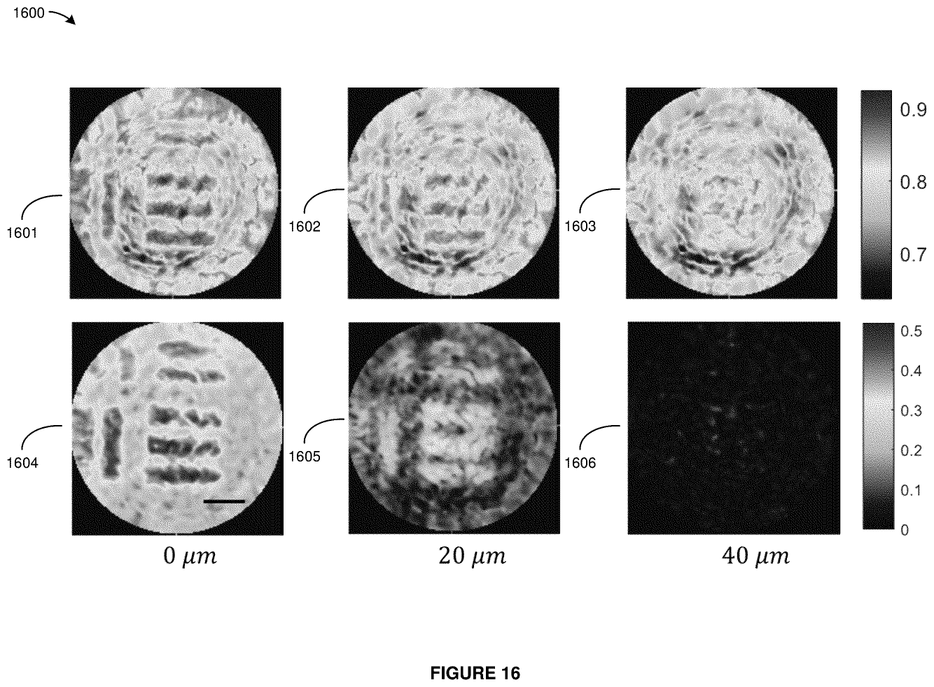

FIG. 16 illustrates environment 1600. Environment 1600 comprises reconstructed images 1601-1606. Reconstructed images 1601-1603 comprises SPI image reconstructions at an image distance of 0, 20, and 40 microns from the object respectively. Reconstructed images 1603-1606 comprises MUSSIC image reconstructions at an image distance of 0, 20, and 40 microns from the object respectively. The FOV shows the first element of the 7^{th} group in the resolution target. The 2D target is moved in steps of 20 μm in the axial direction and away from the fiber distal facet and capture the back-reflected fields from the object at three z-positions. As shown in reconstructed images 1601 and 1604, the target object is visible in both using both the SPI and MUSSIC imaging techniques. As shown in reconstructed images 1602 and 1605, the target object almost disappears in the background already after a movement of 20 μm in the case of MUSSIC reconstructed image 1605. In contrast, SPI reconstruction 1602 carry a significant amount of energy from the sample even after a z-displacement of 40 μm. Hence the MUSSIC approach performs better in rejecting the light out of the image plane. Postprocessing for the MUSSIC images involved regularized TM inversion, bandpass filtering and normalization, as explained in the methods section. Scale bar is 10 μm.

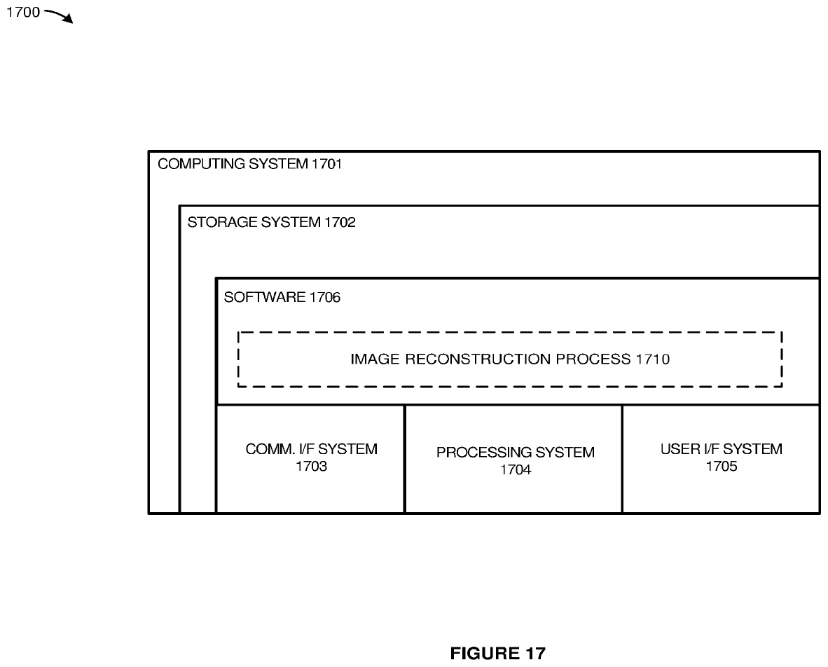

FIG. 17 illustrates environment 1700 which comprises computing system 1701 that is representative of any system or collection of systems in which the various processes, programs, services, and scenarios disclosed herein may be implemented. For example, computing system 1701 may comprise a computing device to perform algorithmic image reconstruction for a MUSSIC microscopy system. Examples of computing system 1701 include computers, servers, controllers, and the like. Computing system 1701 may be implemented as a single apparatus or may be implemented in a distributed manner Computing system 1701 comprises storage system 1702, communication interface system 1703, processing system 1704, interface system 1705, and software 1706. Processing system 1704 is operatively coupled with storage system 1702, communication interface system 1703, and user interface 1705.

Processing system 1704 loads and executes software 1706 from storage system 1702. Software implements image reconstruction process 1710 that is representative of the MUSSIC image reconstruction processes described in the preceding Figures. For example, MUSSIC microscopy system 100 may comprise a computing device configured implement image reconstruction process 1710. When executed by processing system 1704, software 1702 directs processing system 1704 to operate as described herein for at least the various processes, operational scenarios, and sequences discussed in the foregoing implementations. Computing system 1701 may optionally include additional features that are omitted for brevity.

Processing system 1704 comprises a micro-processor and/or other circuitry that retrieves and executes the software from the storage system. Processing system 1704 may be implemented within a single processing device but may also be distributed across multiple processing devices or sub-systems that cooperate in executing program instructions. Examples of processing system 1704 include general purpose CPUs, GPUs, ASICs, FPGAs, logic devices, and the like.

Storage system 1702 comprises computer readable storage media that is readable by processing system 1704 and capable of storing software 1706. Storage system 1702 includes storage media implemented in any method or technology for storage of information like computer readable instructions, data structures, program modules, or other data. Examples of storage media include RAM, read only memory, magnetic disks, optical disks, optical media, flash memory, virtual memory and non-virtual memory, and the like. Storage system 1702 may also include computer readable communication media over which at least some of the software may be communicated internally or externally. Storage system 1702 may be implemented as a single storage device or implemented across multiple co-located or distributed storage devices. Storage system 1702 may comprise additional elements like a controller for communicating with processing system 1704.

Image reconstruction process 1710 may be implemented in program instructions that, when executed by processing system 1704, direct processing system 1104 to operate as described with respect to the preceding Figures. For example, software 1706 may comprise program instructions for implementing image reconstruction process 1710 as described herein. The program instructions include various components or modules that interact to carry out the various processes. The components and/or modules may be embodied in compiled instructions, interpreted instructions, or in some other type of instructions. The components and/or modules may be executed in a synchronous or asynchronous manner, serially or in parallel, in a single threaded environment or multi-threaded, or in accordance with some other execution paradigm. Software 1706 may include additional processes, programs, or components, such as operating systems, virtualization software, or other application software. Software 1706 may also comprise firmware or some other form of machine-readable processing instructions executable by processing system 1704.

Software 1706, when loaded into processing system 1704 and executed, transforms a suitable apparatus, system, or device (of which the computing system is representative) from a general-purpose computing system into a special-purpose computing system customized to perform image reconstruction for a MUS SIC microscopy system. Encoding software 1706 onto storage system 1702 transforms the physical structure of storage system 1702. The specific transformation of the physical structure depends on various factors like the technology used to implement the storage media of storage system 1702 and whether the computer-storage media are characterized as primary or secondary storage. For example, if the computer readable storage media are implemented as semiconductor-based memory, software 1706 transforms the physical state of the semiconductor memory when the program instructions are encoded thereby transforming the state of transistors, capacitors, or other discrete circuit elements constituting the semiconductor memory.

Communication interface system 1703 may include communication connections and devices that allow for communication with other computing systems over communication networks. Examples of the connections and devices that together allow for inter-system communication may include network interface cards, antennas, power amplifiers, RF circuitry, transceivers, and the like. The connections and devices may communicate over communication media like metal, glass, air, or another type of communication media. Communication between computing system 1701 and other computing systems (not shown), may occur over a communication network or networks and in accordance with communication protocols. Examples include intranets, internets, local area networks, wide area networks, wireless networks, and the like.

In conclusion, MUS SIC microscopy through a multimode fiber to enable imaging with optical sectioning, high SNR, and improved resolution has been demonstrated. The improvement in contrast shown is not a fundamental limit and further improvement is possible by increasing the number of pinholes. The cost to pay in exchange is the computational complexity which grows linearly with the number of confocal images, Nₚᵢₙₕₒₗₑₛ .

Besides the challenge of computational complexity, the quality of image reconstruction is limited by several experimental factors. Firstly, the image quality is dependent on the accuracy of the reconstructed virtual distal fields, which is in turn determined by the quality of the inverse estimate of the backward TM. Secondly, we assumed a perfect reflective mirror whose reflection matrix is an identity matrix, for calibrating the backward TM. In practice, some light is lost at the mirror and does not couple back into the fiber. Moreover, the object should be positioned precisely in the plane of the mirror used during the calibration of the backward TM. Deviations may introduce noise to the image reconstruction.

Furthermore, to keep the various MUSSIC microscopy systems simple and robust to thermal and mechanical fluctuations, an internal reference for phase measurements which transform to a non-uniform speckle in the plane of interest with many nulls, also known as blind spots, can be used. The field from these blind spots cannot be recovered, which further degrade the image reconstruction quality. Using complementary reference speckles or an external plane wave reference are possible ways to eliminate the blind spots, although they may require increased measurement time or a more complex setup with phase tracking to account for phase drifts. Bending sensitivity of the fiber is another challenge and any perturbations after calibration may lead to noise in the image reconstruction. MUSSIC microscopy with improved SNR through a multimode fiber could be of practical significance for various microscopy applications in scattering media.

The high SNR capability of MUSSIC microscopy paves a feasible path to coherent imaging and fluorescence imaging Calibration of the multispectral TM of scattering media has been demonstrated. With the help of the multispectral TM, multi-spectral focal spots can be scanned on the proximal side while speckle patterns are projected on the object at the distal end. With knowledge of the distal intensity patterns, the object can be recovered. An advantage of scanning focal spots on the proximal side is that it eliminates the need for coherent backpropagation and enables imaging by solving a simpler intensity-only inverse problem.

A further generalization of the technique can be made by choosing distal illuminations that are not focal spots, but arbitrary speckle patterns. In this case, the backpropagated distal fields E^{d} can be calculated by left multiplying the right-hand side of equation 6 with the illumination matrix, E^{il} and right multiplying it with the conjugate transpose or Tikhonov regularized inverse of E^{il}. Speckle illumination is ideal for compressive sampling and can enable imaging with fewer illumination patterns and shorter data acquisition times. Furthermore, it can also eliminate the need for wavefront shaping if a scanning focal spot field is chosen as input, which only requires a focused beam and a steering mechanism.

Overall, the results demonstrate the capability of MUSSIC microscopy in enabling high SNR and high-resolution imaging through an endoscope for investigating the deep tissue regime. Given the generalized principle of the technique, its application is not limited to the raster scan approach or to multimode fibers and can easily be adapted to other endoscopic probes that might require different excitation and detection paths such as double-clad fibers.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

The included descriptions and figures depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the disclosure. Those skilled in the art will also appreciate that the features described above may be combined in various ways to form multiple embodiments. As a result, the invention is not limited to the specific embodiments described above, but only by the claims and their equivalents.

What is claimed is:

1. A method to image an object located on the distal side of a complex medium, the method comprising:
delivering a wave through the complex medium from the proximal side of the complex medium towards the distal side of the complex medium;
receiving secondary waves that are generated from the object that propagate back through the complex medium;
collecting secondary wave properties on the proximal side of the complex medium at multiple locations; and
implementing a reconstruction algorithm to recover images and/or other properties of the object.

2. The method of claim 1 wherein the complex medium comprises a multimode optical fiber.

3. The method of claim 1 wherein the secondary waves comprise scattered waves from the object.

4. The method of claim 1 wherein the secondary waves comprise fluorescent emissions from the object.

5. The method of claim 1 wherein the secondary waves comprise nonlinear waves generated through interaction of the wave with the object.

6. The method of claim 1 wherein the secondary waves comprise one or more of a second harmonic wave, a multi-photon excitation wave, a Raman wave, or a CARS.

7. The method of claim 1 wherein collecting the secondary wave properties comprises collecting the secondary wave properties by a camera.

8. The method of claim 1 wherein collecting the secondary wave properties comprises collecting the secondary wave properties by one or more point detectors that are shifted in space.

9. The method of claim 1 wherein implementing the reconstruction algorithm to recover images or properties of the object comprises using the forward and/or backward transmission matrix of the complex medium.

10. The method of claim 1 wherein implementing the reconstruction algorithm to recover images or properties of the object comprises performing by digital phase conjugation.

11. The method of claim 1 wherein implementing the reconstruction algorithm to recover images or properties of the object comprises performing optical correlation.

12. The method of claim 1 wherein the waves comprise optical waves.

13. The method of claim 1 wherein the complex medium comprises a shift variant system.

14. The method of claim 1 further comprising spatially and/or temporally modulating the wave to produce focus spots and/or other patterns on the object.

15. The method of claim 1 wherein the wave and/or the secondary wave comprises an optically or digitally phase conjugated wave.

16. A system to image or sense objects, the system comprising:
a complex medium wherein the complex medium comprises at least one of an engineered complex medium or a naturally occurring complex medium;
a wave radiation source configured to transmit waves through the complex medium;
one or more wave modulators configured to modulate the waves transmitted through the complex medium wherein the one or more wave modulators comprise at least one of a spatial wave modulator or a temporal wave modulator;
one or more detectors configured to detect wave properties from secondary waves that propagate back through the complex medium; and
a digital processor configured to reconstruct information based on the secondary wave properties.

17. The system of claim 16 wherein the complex medium comprises at least one of a multimode fiber, metamaterial, a diffractive element, a hologram, a coded aperture, or a stratified element.

18. The system of claim 16 wherein the wave radiation source comprises a laser.

19. The system of claim 16 wherein the one or more modulators comprises a Spatial Light Modulator (SLM).

20. The system of claim 16 wherein the one or more detectors comprise at least one of a camera, a photodetector, a photodetector array, a single photon avalanche diode array, or a photomultiplier tube.

21. The system of claim 16 wherein the one or more modulators comprise a primary Spatial Light Modulator (SLM) configured to modulate the waves transmitted through the complex medium and a secondary SLM configured to modulate the secondary waves that propagate back through the complex medium.

22. The system of claim 16 wherein the complex medium comprises a transmission matrix for the waves transmitted through the complex medium and a secondary transmission matrix for the secondary waves that propagate back through the complex medium.

23. The system of claim 22 wherein the digital processor is configured to reconstruct an image using the transmission matrix and the secondary transmission matrix.

24. The system of claim 22 wherein the digital processor is configured to reconstruct an image using the transmission matrix and the secondary transmission matrix for a nonlinearly generated signal.

25. The system of claim 16 further comprising one or more optical elements positioned between the complex medium and the one or more wave modulators and/or between the complex medium and the one or more detectors wherein the one or more optical elements comprise at least one of a lens, a mirror, a polarizer, a wave plate, a color filter, a spatial filter, a prism, a grating, or a fiber optic device.
